(19) United States
(12) Patent Application Publication
(10) Pub. No.: US 2011/0184269 A1
(43) Pub. Date: Jul. 28, 2011

(54) IMPLANTABLE PROBE

(76) Inventors: Fabien Sauter-Starace, Seyssinet-Pariset (FR); Bibari Olivier, Choicy-Le-Roi (FR); Chabrol Claude, Poisat (FR); Catherine Pudda, Seyssinet-Pariset (FR)

(21) Appl. No.: 13/003,492

(22) PCT Filed: Jul. 10, 2009
(86) PCT No.: PCT/FR2009/000862

§ 371 Date: Mar. 31, 2011

(30) Foreign Application Priority Data
Jul. 11, 2008 (FR) 0803975

(51) Int. Cl. A61B 5/0476; A61N 1/05

(52) U.S. Cl. 600/378; 607/116

(57) ABSTRACT

An implantable probe for acquiring neural signals or for electrically stimulating neurons, the probe comprising: a support (ES) of flexible polymer material; an inorganic substrate (S′) fastened to said support and having thickness that is sufficiently small to present flexibility comparable to that of the support; at least one electrode (EL) carried by said substrate; and a layer (NC) of conductive material deposited by high temperature growth on a surface of said or each electrode and suitable for improving at least one property thereof selected from: electrical properties; biocompatibility properties; and biostability properties. A method of fabricating such a probe, including making electrodes on said inorganic substrate, thinning it, depositing said conductive layer thereon by growth at high temperature, and subsequently transferring the thinned substrate carrying the coated electrodes onto the support of flexible polymer material.

The invention relates to an implantable probe for acquiring neural signals and/or for electrically simulating neurons.

The probe of the invention may be useful in therapeutic applications (blocking epileptic fits by local electrical stimulation, treating Parkinson's disease by deep brain stimulation (DBS)), or diagnostic applications (locating epileptic foci). It may also be used for making direct neural interfaces or “brain-computer” interfaces (BCI), enabling prostheses or motor-driven vehicles to be controlled by measuring brain activity, or indeed for restoring vision or hearing by stimulating the optical or auditory nerve or by stimulating the cortex.

Numerous probes of this type are known in the prior art. Usually they comprise matrices of electrodes made of biocompatible metal on supports of flexible polymer that is also biocompatible. The use of such a flexible support is advantageous in that it serves to minimize the invasiveness of the probe, and consequently the lesions to the parenchyma that it inevitably causes.

The article by K. N. Fountas et al. “Implantation of a closed-loop stimulation in the management of medically refractory focal epilepsy”, Stereotact. Funct. Neurosurg. 2005; 83: pp. 153-158 describes such a probe, and its use in detecting and controlling epileptic fits.

The articles:

E. A. Felton, J. A. Wilson, J. C. Williams, and P. C. Garell “Electrocorticographically controlled brain-computer interfaces using motor and sensory imagery in patients with temporary subdural electrode implants. Report of four cases”, J. Neurosurg. 106 (2006), pp. 495-500; and

G. Schalk, J. Kubanek, K. J. Miller, N. R. Anderson, E. C. Leuthardt, J. G. Ojemann, D. Limbrick, D. W. Moran, L. A. Gerhardt, and J. R. Wolpaw “Decoding two-dimensional movement trajectories using electrocorticographic signals in humans”, J. Neural. Eng. 4 (2007), pp. 264-275;

describe the use of flexible polymer probes designed for locating epileptic foci, in order to provide direct neural interfaces.

Results concerning direct neural interfaces have also been obtained in man using rigid electrode matrices that penetrate into the cerebral cortex, said to be of “Utah” type, and described in particular in document U.S. Pat. No. 7,212,851. Those probes are very invasive, in particular because of the needle shape of the electrodes and because of their level of stiffness.

Implantable probes used in clinical practice and in research are biocompatible, but they present problems of biostability, i.e. their performance degrades over time. They trigger inflammatory responses that lead to them becoming encapsulated in a glial scar; in turn, this encapsulation leads to a progressive degradation of the properties of the probe in terms of sensitivity when measuring neuroelectrical signals and/or effectiveness when stimulating neurons. To minimize that effect, attempts have been made to produce probes that present a minimum amount of invasiveness.

Recent studies have shown that using electrodes having a coating based on carbon nanotubes makes it possible to significantly improve the biocompatibility and the biostability of probes for recording and stimulating neurons: in this respect reference may be made to document U.S. Pat. No. 7,162,308.

Other studies, carried out in vitro, have shown that such a coating also serves to improve the electrical properties of implantable probes. Reference may be made in particular to the following publications:

Gabay et al. “Electrochemical and biological properties of carbon nanotube based multi-electrode arrays”, Nanotechnology 18 (2007), 035201; and

Wang et al. “Neural stimulation with a carbon nanotube microelectrode array”, Nano Lett., Vol. 6, No. 9, 2006.

One difficulty lies in the fact that carbon nanotubes are grown at temperatures that are relatively high (greater than about 400° C.), and that are incompatible with using support elements made of flexible polymer material. By way of comparison, polyimides do not withstand temperatures higher than 380° C. Thus, in accordance with above-mentioned document U.S. Pat. No. 7,162,308, electrode arrays including such a coating need to be made on inorganic supports that are rigid, in particular that are made of metal.

Unfortunately, using a rigid support makes the probe more invasive, and thus more likely to give rise to a response on the part of the organism, thereby canceling the advantage obtained by the carbon nanotube coating in terms of biocompatibility and biostability.

The article by Allen Ashanté et al. “Flexible microdevices based on carbon nanotubes”, 2006 J. Micromech. Microeng. 16, pp. 2722-2729 describes an implantable probe comprising a support element made of flexible polymer and electrodes having carbon nanotubes adhesively bonded thereon. That technique overcomes the difficulty posed by the incompatibility between polymer supports and the high temperatures needed for growing nanotubes.

Nevertheless, using adhesive with nanotubes reduces their developed surface area, their conductivity, and the quality of their electrical contact with the electrodes.

An object of the invention is thus to provide a remedy to the above-mentioned drawbacks of implantable probes known in the prior art.

More precisely, the invention seeks to provide implantable probes that present good electrical, biocompatibility, and biostability properties, while also being of minimum invasiveness.

According to the invention, such an object is achieved by an implantable probe for acquiring neural signals or for electrically stimulating neurons, the probe comprising: a support of biocompatible flexible polymer material provided with conductive tracks; and at least one electrode carried by said support and electrically connected to said conductive tracks; the probe being characterized in that it also comprises: an inorganic substrate that is insulating or semiconductive, fastened to said support and of thickness that is sufficiently small to present flexibility comparable to the flexibility of the support, said electrode(s) being deposited on said substrate; and a layer of conductive material, deposited by high temperature growth on a surface of said or each electrode.

Advantageously, said layer deposited on a surface of said or each electrode may be selected in such a manner as to improve at least one property thereof selected from: electrical properties, biocompatibility properties, and biostability properties.

In particular embodiments of the invention:

Said layer deposited on a surface of said or each electrode may be a nanostructured layer constituted by a material selected from: carbon nanotubes; carbon nanofibers; metal nanowires, in particular made of gold, platinum, or ruthenium; polypirrole nanowires; iridium oxide; platinum black; and doped diamond.

In a variant, said layer deposited on a surface of said or each electrode may be a layer of optionally-nanostructured doped diamond.

Said electrode may include a layer of catalyst metal adapted to enhance high temperature growth of said conductive layer.

Said inorganic substrate may be selected from a substrate of intrinsic or doped silicon, of glass, of borosilicate (pyrex), or of silica.

Said inorganic substrate may present thickness lying in the range 10 micrometers (μm) to 50 μm, and preferably about 30 μm.

The probe may include a plurality of electrodes forming a matrix. Under such circumstances, said inorganic substrate may be subdivided into chips, each carrying one or more of said electrodes.

The invention also provides a method of fabricating a probe according to any preceding claim, comprising the steps consisting in:

a) depositing at least one electrode on a front face of an insulating or semiconductive inorganic substrate;

b) thinning said substrate by abrading a rear face that is opposite from said front face;

c) depositing a layer of conductive material on a face of said or each electrode by high temperature growth, said layer being adapted in particular for improving the biocompatibility of said electrode; and

d) transferring the thinned substrate onto a support of biocompatible flexible polymer material provided with conductive tracks, while ensuring electrical connection between said conductive tracks an the electrode(s) of the substrate.

In particular implementations of the invention:

The method may also include a step a′) consisting in securing the substrate to a backing plate (P) prior to thinning it, in order to facilitate handling, and a step b′) consisting in separating the thinned substrate from the backing plate prior to depositing said layer of conductive material.

A plurality of electrodes may be made on a common substrate, the method also including a step of cutting said substrate to subdivide it into a plurality of chips, each chip carrying one or more electrodes.

Said step c) of high temperature deposition of a layer of conductive material may be performed at a temperature higher than 400° C. and preferably lying in the range 550° C. to 850° C.

Said step a) of depositing at least one electrode on said substrate may comprise:

a1) depositing an adhesion layer on the surface of said substrate so as to avoid forming a Schottky barrier between the substrate and the electrode(s);

a2) depositing a main conductive layer on said adhesion layer, thereby constituting the body of the or each electrode; and

a3) depositing a catalytic conductive layer on said main conductive layer, the catalytic conductive layer being adapted to encourage high temperature growth of said conductive layer.

The method may include a step c′) of depositing a conductive layer on said rear face of the substrate so as to enable the electrodes to be electrically connected to tracks provided on said substrate of biocompatible flexible polymer material.

Other characteristics, details, and advantages of the invention appear on reading the description made with reference to the accompanying figures given by way of example and in which:

FIGS. 1, 2a, 2b, 3a, 3b, and 3c show different steps of a method of fabricating an implantable probe in accordance with the invention;

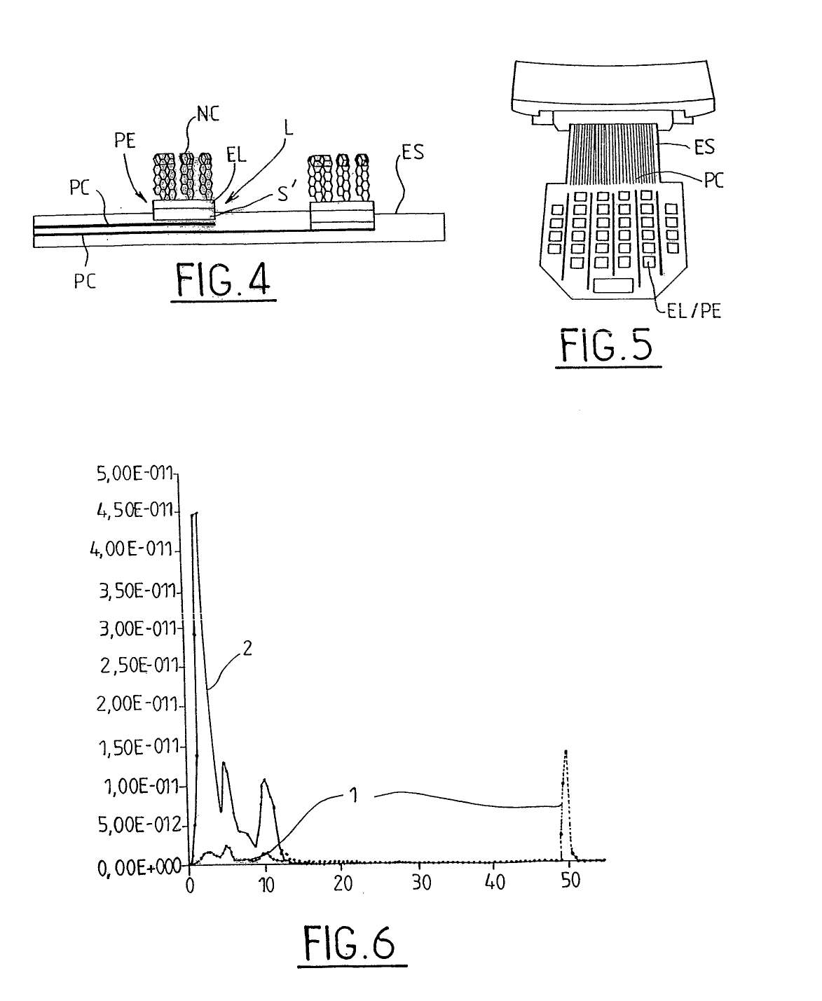

FIGS. 4 and 5 are section and elevation views respectively showing such a probe; and

FIG. 6 is a graph showing the improvement in the performance of the implantable probe that can be obtained by means of the invention.

The fabrication of an implantable probe of the invention begins by making a matrix of electrodes on a biocompatible inorganic substrate that is adapted to withstand relatively high temperatures (several hundreds of degrees). Such a substrate, identified by reference S in FIG. 1, may advantageously be made of silicon, either intrinsic or doped to make it conductive. In a variant it could equally well be made of glass (in particular of pyrex glass) or of silica (SiO₂). The thickness of the substrate is generally of the order of a few hundreds of micrometers, thereby making it very rigid. Typically, silicon wafers are used that have a diameter of 100 millimeters (mm) and thickness lying in the range 300 μm to 525 μm, or a diameter of 200 mm and thickness lying in the range 500 μm to 725 μm.

After deoxidizing the surface of the substrate, various deposits of metal are made on its front face F₁ in order to fabricate the electrodes. Typically, three deposits are needed:

a first deposit for making an adhesion layer C₁;

a second deposit for making a main layer C₂ constituting the bodies of the electrodes; and

a third deposit of a thin layer C₃ of metal or of catalytic alloy encouraging the growth of a coating layer for improving the electrical properties and/or the biocompatibility of the probe (e.g. a layer of carbon nanotubes).

These deposits may be made by conventional sputtering or evaporation techniques.

In a preferred embodiment of the invention, the adhesion layer C₁ is made of titanium Ti and presents thickness of the order of 20 nanometers (nm). During the subsequent step, at high temperature, of growing the surface coating of the electrodes, this adhesion layer combines with the silicon of the substrate in application of the following reaction:

Ti+2Si→TiSi₂

thus serving to avoid forming a Schottky barrier between the substrate and the electrodes, and preventing diffusion phenomena.

Thereafter, a main layer C₂ of titanium nitride TiN and of significantly greater thickness (of the order of 200 nm) is deposited on the adhesion layer to constitute the bodies of the electrodes. In a variant, it is possible to deposit a layer of titanium and subsequently to proceed with nitriding it.

Thereafter a thin (1 nm to 5 nm) layer C₃ of metal or of catalytic alloy is deposited on the main layer C₂. By way of example, the catalyst may be constituted by Ni, Ni₈₀Fe₂₀, Fe, Co, Al, Mo, Pd, or an alloy of these metals.

It should be observed that FIG. 1 is not to scale, the thicknesses of the metal layers being greatly exaggerated compared with the thickness of the substrate.

Finally, cutting and/or dry or wet etching operations are performed in order to separate the electrodes. Cutting also serves to subdivide the substrate S into individual chips, each including one or more electrodes. Under such circumstances, the electrodes on a given chip are separated from one another by regions in which the metal layers have been removed by etching.

In FIG. 1, reference D indicates pre-cutting lines in the substrate S, while reference G indicates a region from which the coating C₁-C₃ is to be removed by etching in order to separate the electrodes The chips defined by the cutting lines D are preferably square or rectangular, with a side of length lying in the range about 100 μm to a few millimeters. The electrodes typically present characteristic dimensions (side or diameter) lying in the range 10 μm to 100 μm for microelectrodes, and preferably in the range 30 μm to 40 μm. For matrices of surface electrodes (for electrocorticograms or electroencephalograms) the preferred characteristic dimensions lie in the range 400 μm to 4 mm, and preferably in the range 1 mm to 2 mm.

The microelectrodes, i.e. the electrodes of characteristic dimensions smaller than one millimeter, present a particular advantage since they make it possible to acquire unit neural signals.

It may be observed that the cutting D of the substrate S is only partial, extending to no more than a depth of a few tens of micrometers.

As explained above, the substrate S presents thickness of a few hundreds of micrometers; consequently, it is rigid. A thinning operation, down to a thickness of the order of 30 μm (more generally lying in the range 10 μm to 50 μm approximately) enables it to be made supple and flexible like the support of polymer material onto which it is to be fastened.

In accordance with the invention, substrate thinning is performed by sticking the front face F₁ of the substrate to a backing plate P, referred to as a “handle”, and then machining the rear face F₂.

The handle P, preferably made of pyrex glass, presents thickness that is at least comparable with the thickness of the non-thinned substrate S. Its role is to enable the thinned substrate to be handled easily and to avoid any untimely breaking thereof. The substrate S and its handle P are bonded together with a heat-sensitive or photo-sensitive adhesive ST (called “sticky” if it is a laminated glue), so as to make separation easy without needing to exert excessive forces on the thinned substrate.

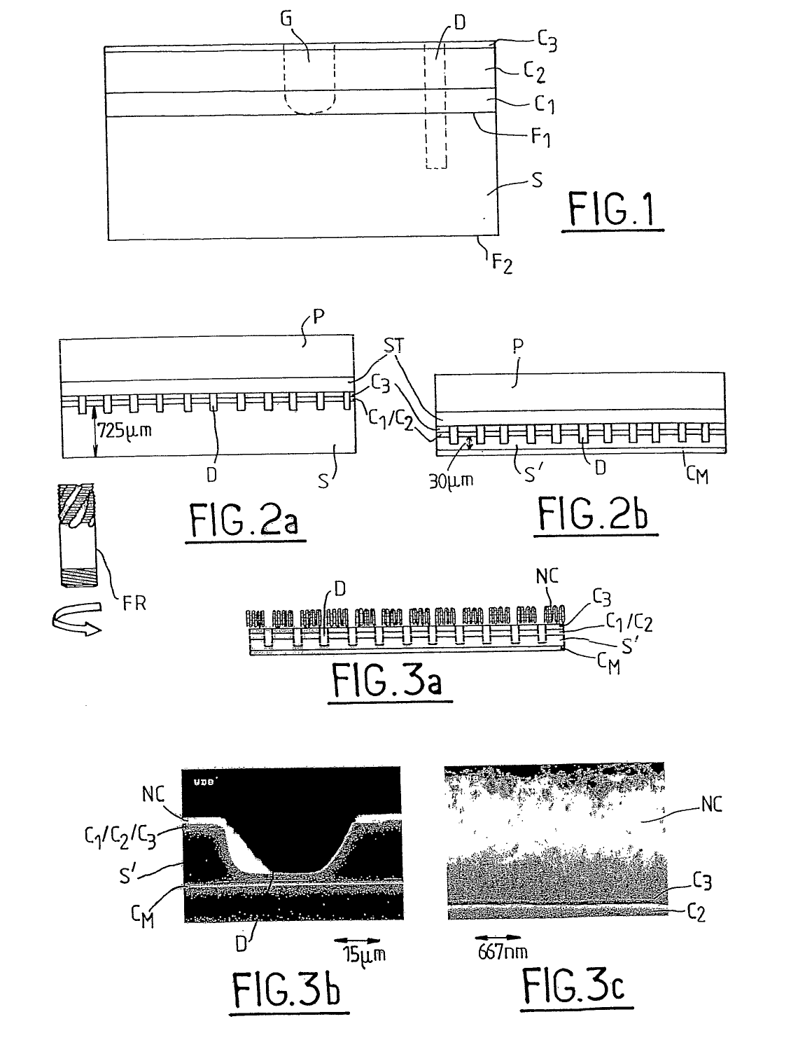

The thinning operation, shown in FIGS. 2a and 2b, may be performed in a plurality of steps in order to optimize both the speed of thinning and the uniformity and thickness of the thinned substrate. For example, it is possible to use a first step of mechanical machining with a cutter FR at a speed lying in the range 80 micrometers per minute (μm/min) to 120 μm/min until a residual thickness of 50 μm is reached, followed by a second step of abrasion by ion bombardment at a speed of about 40 μm/min; the last micrometers of the thickness are removed by a third step of dry or wet etching at a speed of 2 μm/min to 10 μm/min.

It should be observed that the cutting lines D do not extend through the entire thickness of the thinned substrate S′, which thus retains its structural integrity.

Thereafter, a layer of metal CM is deposited on the rear face F₂ of the thinned substrate S′. If the substrate is insulating (glass, silica, intrinsic silicon) then vias are also formed in order to connect the layer on the rear face to the electrodes deposited on the front face D₁. This is not necessary if the substrate is sufficiently conductive.

Thereafter, the handle P is separated from the thinned substrate S′ to expose its front face D₁, and thus enable a layer of carbon nanotubes to be deposited by high temperature growth.

In principle, it is possible to envisage depositing the metal layers C₁-C₃ after thinning the substrate, however the fragility of the thinned substrate makes this variant more difficult to implement.

This growth is performed by methods known in the prior art, in particular as described in above-mentioned document U.S. Pat. No. 7,162,308, using a carbon-containing gas such as a mixture of acetylene and hydrogen at a temperature of about 650° C.

FIG. 3a is a diagrammatic section view of a thinned substrate S′ covered in a layer NC of carbon nanotubes. FIG. 3b is an image obtained by a scanning electron microscope giving a detailed view of the FIG. 3a substrate, and showing particularly clearly the “chocolate bar” cutting D that serves to keep the chips connected to one another until the cleaving operation that precedes transfer onto the matrix. FIG. 3c is a scanning electron microscope image obtained at greater magnification showing the layer or “carpet” of carbon nanotubes deposited on the electrodes of the thinned substrate S′. Scales are marked graphically on FIGS. 3b and 3c.

The layer of carbon nanotubes presents thickness lying in the range 1 μm to 10 μm, with 2 μm being a typical value.

After the carbon nanotubes have been grown, the various chips making up the substrate S′ are separated by breaking along the cutting lines D between them. It is not essential to separate all of the chips: some of them may remain grouped together, in particular if the substrate is dielectric and thus serves to provide insulation between the various electrodes.

Thereafter, the chips or sets of chips are taken by means of an appropriate tool or clamp, taking care to avoid damaging the coating CN since it is very fragile, and they are placed on a support element ES made of flexible polymer, that is electrically insulating and biocompatible, so as to constitute the body of the implantable probe. Amongst the polymer materials that are suitable for implementing the invention, mention may be made of benzocyclobutenes (BCB), polyimides, and polyisoindroquinazorindiones (PIQ).

As shown in FIG. 4, the support element ES is provided with conductive tracks PC made using microfabrication techniques that are themselves known, which tracks are buried over the major portions of their length and exposed solely in housings L provided for receiving the chips obtained from the thinned substrate S′. As shown in FIG. 4, these tracks may be deposited at a plurality of levels.

The chips PE carrying the electrodes EL coated in carbon nanotubes are deposited on the support element ES in said housings L. Mechanical and electrical connection is achieved by means of a biocompatible epoxy adhesive made conductive by adding a filler of metal or based on carbon, or by soldering using an alloy that melts at low temperature (e.g. Au/Sn). The thickness of the adhesive layer or of the solder (not shown in the figures) must be adapted to conserve good flexibility for the chip/support assembly while also avoiding delamination during the welding step or the adhesive application step. A filled adhesive presents the advantage of being capable of being cured at ambient temperature. The use of solder makes it possible to obtain smaller thickness and better electrical contact, but is more complex to implement.

For reasons of biocompatibility, a cord of epoxy resin (not shown in the figures) is formed around each chip to provide sealing. Tests with two-component and UV epoxy sealing adhesives have given good results depending on the type of chemistry and nanotubes used. The solution that is the simplest to implement is a two-component adhesive applied to hydrophobic chips so as to avoid wetting the sensitive surface.

The electrodes transferred onto the support element ES form a one- or two-dimensional matrix that may be regular or otherwise. They are connected to an appliance for detecting neuron activity and/or for generating stimulation pulses via the conductive tracks PC.

The invention is described with reference to a particular embodiment, however numerous variants may be envisaged.

For example, the implantable probe may have a very wide variety of shapes and dimensions. FIG. 5 shows a probe made from a flexible printed circuit. In this figure, there can be seen the metal conductive tracks on polymer insulation (kapton), the base of the probe, and its connector (in the background). The article by Karen C. Cheung et al. “Flexible polyimide microelectrode array for in vivo recordings and current source density analysis”, Biosensors and Bioelectronics 22 (2007), pp. 1783-1790, and also document EP 1 932 561, describe probe structures that may be suitable for implementing the invention. More precisely, the probes are made on the basis of a polyimide layer deposited on a sacrificial layer. The conductive elements are made of a biocompatible metal, such as platinum.

The carbon nanotubes constituting the coating of the electrodes may be single- or multiple-walled, or may present a “bamboo” structure. In addition, said coating need not be based on carbon nanotubes, but on other nanostructures such as carbon nanofibers, doped silicon nanowires, or metal wires (gold, platinum, ruthenium), or indeed wires made of conductive polymer (polypyrroles), nanostructures made of iridium oxide, or nanostructured platinum (platinum black).

A particularly advantageous variant is represented by using a coating of doped diamond, in particular boron-doped diamond, that is optionally nanostructured. In this context, reference may be made to the article by M. Bonnauron et al., Diamond Relat. Mater. (2008), doi: 10.1016/j.diamond.2007.12.065. Under such circumstances, it is possible alternatively to deposit diamond on the front face of the substrate before thinning.

All of these coating materials present two points in common: they can only be deposited at “high” temperature, i.e. at a temperature higher than about 400° C., and in any event at a temperature that cannot be tolerated by the biocompatible flexible polymers that are likely to be used for making the support element ES; and they serve to improve the electrical and/or the biocompatibility/ biostability properties of the electrodes.

Concerning biocompatibility/biostability, in vitro tests have shown that on electrodes carrying a coating of carbon nanotubes, the agglomeration of glial cells (“glios”) covering the electrodes is considerably reduced compared with traditional metal electrodes. Furthermore, during such tests, it has been observed that a much larger quantity of neurons are formed in the vicinity of and at the surface of electrodes of the invention, likewise in comparison with prior art electrodes of comparable area; this is due to the low cytotoxicity of the coating of carbon nanotubes. Thus, the electrodes of the invention present properties of biocompatibility and of biostability that are better than those of prior art electrodes.

Amongst the electrical properties that may be improved (and more particularly, increased), mention may be made in particular of the following:

The electrical conductance of the electrodes.

The modulus of their complex admittance in contact with neural tissue, or surface admittance. Thus, for example, for signals at a frequency lying in the range 10 hertz (Hz) to 3 kilohertz (kHz) the contact impedance |Z_{surf}| (the reciprocal of admittance) relative to a unit area lies in the range 10 ohm square centimeters (Ω.cm₂) to 2×10₃ Ω.cm₂ for electrodes made of TiN. In contrast, |Z_{surf}| remains in the range 1.4 Ω.cm₂ to 21.5 Ω.cm₂ for electrodes provided with a coating of carbon nanotubes. This results in better signal resolution in vivo at low frequency.

The interface capacitance that, by way of example, goes from 5×10₄ farads per square centimeter (F/cm₂) for TiN electrodes to 1.3×10₂ F/cm₂ for electrodes provided with a coating of carbon nanotubes, i.e. an increase of about two orders of magnitude. The higher the interface capacitance, the greater the quantity of charge that can be injected into neural tissue for a stimulation signal of given (voltage) amplitude.

The charge injection limit, an electrochemical property defined as the maximum quantity of charge that an electrode can inject before reaching the electrolysis potential of water.

FIG. 6 plots the power of the neuroelectrical signal as recorded four months after implantation by a probe having TiN electrodes (curve 1) and for a probe having electrodes carrying a carbon nanotube coating made in accordance with the invention. The abscissa axis plots frequency in Hz and the ordinate axis plots signal power on a linear scale (curve 2). It can be seen that the signal acquired by the probe of the invention presents better resolution and greater intensity (by more than one order of magnitude), in particular at low frequencies.

1. An implantable probe for acquiring neural signals or for electrically stimulating neurons, the probe comprising:
(a) a support (ES) of biocompatible flexible polymer material provided with conductive tracks (PC); and
(b) at least one electrode (EL) carried by said support and electrically connected to said conductive tracks;
the probe being characterized in that it also comprises:
(c) an inorganic substrate (S′) that is insulating or semiconductive, fastened to said support and of thickness that is sufficiently small to present flexibility comparable to the flexibility of the support, said electrode(s) being deposited on said substrate; and
(d) a layer (NC) of conductive material, deposited by high temperature growth on a surface of said or each electrode.

2. A probe according to claim 1, wherein said layer (NC) deposited on a surface of said or each electrode is selected in such a manner as to improve at least one property thereof selected from: electrical properties, biocompatibility properties, and biostability properties.

3. A probe according to claim 1, wherein said layer (NC) deposited on a surface of said or each electrode is a nanostructured layer constituted by a material selected from:
carbon nanotubes;
carbon nanofibers;
metal nanowires, in particular made of gold, platinum, or ruthenium;
polypirrole nanowires;
iridium oxide;
platinum black; and
doped diamond.

4. A probe according to claim 1, wherein said layer deposited on a surface of said or each electrode is a layer of doped diamond.

5. A probe according to claim 1, wherein said electrode includes a layer (C3) of catalyst metal adapted to enhance high temperature growth of said conductive layer.

6. A probe according to claim 1, wherein said inorganic substrate is selected from a substrate of intrinsic or doped silicon, of glass, of pyrex glass, or of silica.

7. A probe according to claim 1, wherein said inorganic substrate presents thickness lying in the range 10 μm to 50 μm.

8. A probe according to claim 1, including a plurality of electrodes forming a matrix.

9. A probe according to claim 8, wherein said inorganic substrate is subdivided into chips (PE) each carrying one or more of said electrodes.

10. A method of fabricating a probe according to claim 1, the method comprising the steps consisting in:
(a) depositing at least one electrode (EL) on a front face (F1) of an insulating or semiconductive inorganic substrate;
(b) thinning said substrate by abrading a rear face (F2) that is opposite from said front face;
(c) depositing a layer of conductive material (NC) on a face of said or each electrode by high temperature growth; and
(d) transferring the thinned substrate onto a support (ES) of biocompatible flexible polymer material provided with conductive tracks (PC), while ensuring electrical connection between said conductive tracks and the electrode(s) of the substrate.

11. A method according to claim 10, also including a step a′) consisting in securing the substrate to a backing plate (P) prior to thinning it, in order to facilitate handling, and a step b′) consisting in separating the thinned substrate from the backing plate prior to depositing said layer of conductive material.

12. A method according to claim 10, wherein a plurality of electrodes are made on a common substrate, the method also including a step of cutting said substrate to subdivide it into a plurality of chips (PE), each chip carrying one or more electrodes.

13. A method according to claim 10, wherein said step c) of high temperature deposition of a layer of conductive material is performed at a temperature higher than 400° C. and preferably lying in the range 550° C. to 850° C.

14. A method according to claim 10, wherein said step a) of depositing at least one electrode on said substrate comprises:
(a1) depositing an adhesion layer (C1) on the surface of said substrate so as to avoid forming a Schottky barrier between the substrate and the electrode(s);
(a2) depositing a main conductive layer (C2) on said adhesion layer, thereby constituting the body of the or each electrode; and
(a3) depositing a catalytic conductive layer (C3) on said main conductive layer, the catalytic conductive layer being adapted to encourage high temperature growth of said conductive layer (NC).

15. A method according to claim 10, also including a step c′) of depositing a conductive layer (CM) on said rear face of the substrate so as to enable the electrodes to be electrically connected to tracks provided on said substrate of biocompatible flexible polymer material.
